(19) United States
(12) Patent Application Publication
(10) Pub. No.: US 2026/0232746 A1
(43) Pub. Date: Aug. 13, 2026

(54) Composition Comprising a Sceletium Extract and Uses Thereof

(71) Applicant: Stellenbosch University, Stellenbosch (ZA)

(72) Inventors: Nokwanda Pearl Makunga, Stellenbosch (ZA); Kaylan Reddy, Stellenbosch (ZA); Catherine Hart Kaschula, Stellenbosch (ZA)

(73) Assignee: Stellenbosch University, Stellenbosch (ZA)

(21) Appl. No.: 19/469,705

(22) PCT Filed: Mar. 28, 2023
(86) PCT No.: PCT/ZA2023/050017

§ 371 Date: Sep. 26, 2025

(51) Int. Cl. A61K 36/185; A61K 31/137; A61K 31/404; A61K 31/475; A61P 25/24

(52) U.S. Cl. CPC A61K 36/185; A61K 31/137; A61K 31/404; A61K 31/475; A61P 25/24; A61K 2236/33; A61K 2236/53

(57) ABSTRACT

The invention provides a composition comprising an extract of a plant from the genus Sceletium, wherein the extract includes the alkaloids sceletium A4 (sceletium A4 1), an isomer of sceletium A4 (sceletium A4 2) and epimesembranol, and wherein the content of sceletium A4 1 is not less than about 0.9% (w/w) of the total alkaloid content of the extract, the content of sceletium A4 2 is not less than about 1% (w/w) of the total alkaloid content of the extract, and the content of epimesembranol is not less than about 1% (w/w) of the total alkaloid content of the extract. The composition can be used in medicine, such as for preventing, treating or reducing depression, anxiety, stress, stress-induced fatigue or mood, or diseases, disorders or conditions caused by these.

### FIELD OF THE INVENTION

The invention relates to a composition comprising a Sceletium extract and to the use of the composition in reducing anxiety, depression or stress or in improving cognitive functioning or mood.

### BACKGROUND TO THE INVENTION

Stress, anxiety and depression pose a risk to the ability to lead a meaningful life. The World Health Organisation has reported a 25% increase in anxiety and depression worldwide since the emergence of Covid-19. However, the mental health crisis is not a new one and depression and anxiety are the leading cause of disability globally. In addition, these mental health conditions often co-occur.

The monoamine hypothesis of depression states that depression arises from a disbalance and depletion in the levels of the neurotransmitters serotonin, noradrenaline, and/or dopamine. Antidepressants act to improve mood by increasing the levels of these neurotransmitters. However, many medications have unpleasant side-effects and as a result, many people suffering from low mood, depression or anxiety may choose to go without medical treatment.

There is thus a need for new medications that are effective at relieving mental health symptoms and improving quality of life without the burden of side effects.

Sceletium tortuosum (L.) N.E. Br (Mesembryanthemaceae) is one of South Africa's most researched plant species for its therapeutic role on the central nervous system. It promotes relaxation and mood elevation and is used for its anxiolytic effects. Of the eight species in the genus, only S. tortuosum (commonly known as ‘kougoed’, ‘kanna’ or ‘channa’) is used in the phytopharmaceutics industry. The mood-elevating activity of S. tortuosum has been attributed to mesembrine, mesembrenol and mesembrenone acting as serotonin reuptake inhibitors (e.g. U.S. Pat. Nos. 8,552,051, 8,980,338 and 9,381,220). Thus, commercial plant extracts of S. tortuosum are usually prepared from S. tortuosum plants having high levels of one or more of these alkaloids. However, some compositions claiming to include S. tortuosum extracts have been reported to have little or no benefit in improving mood disorders or depression or in relieving stress.

The applicant has now identified ecotypes of S. tortuosum, which are high in other mesembrine-type alkaloids (i.e. alkaloids related in structure but different to mesembrine, mesembrenol and mesembrenone), and extracts of these ecotypes appear to be superior at increasing serotonin and noradrenaline levels compared to industry samples, in the regions of the prefrontal cortex, the hypothalamus and the striatum of mice.

### SUMMARY OF THE INVENTION

According to a first aspect of the invention, there is provided a composition comprising an extract of a plant from the genus Sceletium,
wherein the extract comprises sceletium A4 (sceletium A4 1), an isomer of sceletium A4 (sceletium A4 2) and epimesembranol;
and wherein:
the content of each of the sceletium A4 alkaloids is not less than about 0.9% (w/w) of the total alkaloid content of the extract; and
the content of epimesembranol is not less than about 1% (w/w) of the total alkaloid content of the extract.

The content of sceletium A4 1 may be not less than about 5% (w/w) of the total alkaloid content of the extract.

The content of sceletium A4 2 may be not less than about 5% (w/w) of the total alkaloid content of the extract.

The content of epimesembranol may be not less than about 7% (w/w) or not less than about 20% (w/w) of the total alkaloid content of the extract.

The combined content of sceletium A4 1 and sceletium A4 2 may be at least about 5% (w/w) of the total alkaloid content of the extract.

The extract may further comprise the alkaloids Δ7-mesembrenone, mesembrenol, mesembranol, mesembrenone, mesembrine, O-demethylmesembrenone, O-demethylmesembrine, dihydrojoubertamine, O-acetylmesembrenol and/or epimesembrenol, or any combination thereof.

The combined content of mesembrenol, mesembranol, epimesembranol, O-acetyl-mesembrenol and epimesembrenol may be at least about 5% (w/w) of the total alkaloid content of the extract, and may even be at least about 50% (w/w) of the total alkaloid content of the extract.

According to a second aspect of the invention, there is provided a composition substantially as described above, for use as a medicament.

According to a third aspect of the invention, there is provided a composition substantially as described above, for use in preventing, treating or reducing diseases or conditions selected from the group consisting of depression, anxiety, stress and stress-induced fatigue or for enhancing cognitive functioning or mood.

According to a further aspect of the invention, there is provided a use of a composition substantially as described above in the manufacture of a medicament for use in a method of treating, reducing or preventing diseases or conditions selected from the group consisting of depression, anxiety, stress and stress-induced fatigue or for enhancing cognitive functioning or mood.

According to yet a further aspect of the invention, there is provided a method of treating, reducing or preventing diseases or conditions selected from the group consisting of depression, anxiety, stress and stress-induced fatigue or for enhancing cognitive functioning or mood, the method comprising administering an effective amount of a composition substantially as described above to a subject in need thereof.

### BRIEF DESCRIPTION OF THE FIGURES

FIG. 1A: LC-MS results of a sample of an S. totuosum plant, designated “DR”, containing, among others, the alkaloids sceletium A4 (sceletium A4 1) (retention time 4.8 min), an isomer of sceletium A4 (sceletium A4 2) (retention time 5.1 min, and epimesembranol.

FIG. 1B: LC-MS results of a sample of an S. totuosum plant, designated “3K”, containing, among others, the alkaloids sceletium A4 (sceletium A4 1), an isomer of sceletium A4 (sceletium A4 2) and epimesembranol.

FIG. 1C: LC-MS results of an industry sample containing an extract of an S. totuosum plant.

FIG. 2: Graphical representation of the comparison of the concentration of monoamine serotonin measured in the prefrontal cortex (A), hippocampus (B) and striatum (C) within five different exposure groups (n=12). The error bars represent means with 95% confidence intervals. Kruskal-Wallis H test followed by Dunn's procedure with Bonferroni adjustments for multiple comparisons p<0.05; p<0.01**, ***p<0.001, PFC: prefrontal cortex, HIP: hippocampus, STR: Striatum, Ctrl: Control, EtOH 5% Ctrl: Ethanol 5% control, 10 mg/kg daily dose of industry sample, 3K sample or DR sample.

FIG. 3: Graphical representation of the comparison of the concentration of monoamine noradrenaline measured in the prefrontal cortex (A), hippocampus (B) and striatum (C) within the five different exposure groups (n=12). The error bars represent means with 95% confidence intervals. Kruskal-Wallis H test followed by Dunn's procedure with Bonferroni adjustments for multiple comparisons *p<0.05, **p<0.01, ***p<0.001, ****p<0.0001. PFC: prefrontal cortex, HIP: hippocampus, STR: Striatum, Ctrl: Control, EtOH 5% Ctrl: Ethanol 5% control. 10 mg/kg daily dose of industry sample, 3K sample or DR sample.

FIG. 4: Graphical representation of the comparison of the concentration of monoamine dopamine measured in the prefrontal cortex (A), hippocampus (B) and striatum (C) within the five different exposure groups (n=12). The error bars represent means with 95% confidence intervals. Kruskal-Wallis H test followed by Dunn's procedure with Bonferroni adjustments for multiple comparisons p<0.05. PFC: prefrontal cortex, HIP: hippocampus, STR: Striatum, Ctrl: Control, EtOH 5% Ctrl: Ethanol 5% control. 10 mg/kg daily dose of industry sample, 3K sample or DR sample.

FIG. 5: Graphical representation of the comparison of the concentration of monoamine GABA measured in the prefrontal cortex (A), hippocampus (B) and striatum (C) within the five different exposure groups (n=12). The error bars represent means with 95% confidence intervals. Kruskal-Wallis H test followed by Dunn's procedure with Bonferroni adjustments for multiple comparisons *p<0.05, **p<0.01, ***p<0.001, ****p<0.0001. GABA: gamma-aminobutyric acid PFC: prefrontal cortex, HIP: hippocampus, STR: Striatum, Ctrl: Control, EtOH 5% Ctrl: Ethanol 5% control. 10 mg/kg daily dose of industry sample, 3K sample or DR sample.

### DETAILED DESCRIPTION OF THE INVENTION

The invention provides a composition comprising an extract of a plant from the genus Sceletium, wherein the extract includes the alkaloids sceletium A4 (sceletium A4 1), an isomer of sceletium A4 (sceletium A4 2), epimesembranol, epimesembrenol and/or O-demethylmesembrine. Preferably, the extract includes the alkaloids sceletium A4 (sceletium A4 1), an isomer of sceletium A4 (sceletium A4 2) and epimesembranol, wherein the content of sceletium A4 1 is not less than about 0.9% (w/w) of the total alkaloid content of the extract, the content of sceletium A4 2 is not less than about 1% (w/w) of the total alkaloid content of the extract, and the content of epimesembranol is not less than about 1% (w/w) of the total alkaloid content of the extract.

The Sceletium extract can be from a plant of any of the species in the Sceletium genus, i.e. Sceletium tortuosum, Sceletium strictum, Sceletium rigidum, Sceletium emarcidum, Sceletium exaltum, Sceletium crassicaule, Sceletium expansum or Sceletium varians. In one embodiment, the extract is from Sceletium tortuosum.

In some embodiments, the content of sceletium A4 1 is not less than about 2% (w/w), not less than about 3% (w/w), not less than about 4% (w/w) or not less than about 5% (w/w) of the total alkaloid content of the extract.

In some embodiments, the content of sceletium A4 2 is not less than about 2% (w/w), not less than about 3% (w/w), not less than about 4% (w/w), not less than about 5% (w/w), not less than about 6% (w/w) or not less than about 7% (w/w) of the total alkaloid content of the extract.

In some embodiments, the combined content of sceletium A4 and its isomer (i.e. sceletium A4 1 and sceletium A4 2) is at least about 5% (w/w), at least about 6% (w/w), at least about 7% (w/w) or at least about 8% (w/w) of the total alkaloid content of the extract.

In some embodiments, the content of epimesembranol is not less than about 2% (w/w), not less than about 5% (w/w), not less than about 7.5% (w/w), not less than about 10% (w/w), not less than about 15% (w/w) or not less than about 20% (w/w) of the total alkaloid content of the extract.

The extract can also include any one or more of the following alkaloids: Δ7-mesembrenone, mesembrenol, mesembranol, mesembrenone, mesembrine, O-demethylmesembrenone, O-demethylmesembrine, dihydrojoubertamine, O-acetylmesembrenol and epimesembrenol. In particular, the extract can include mesembrenol, O-demethylmesembrine and epimesembranol. The content of each of these alkaloids is typically not less than about 0.1% (w/w), more typically not less than about 1% (w/w) of the total alkaloid content of the extract. In one embodiment, the extract includes all of these alkaloids.

In one embodiment, the extract includes sceletium A4 and/or isomers thereof and any one or more of the alkaloids selected from the group consisting of epimesembranol, Δ7-mesembrenone, mesembrenol, mesembranol, mesembrenone, mesembrine, O-demethylmesembrenone, O-demethylmesembrine, dihydrojoubertamine, O-acetylmesembrenol and epimesembrenol.

In one embodiment, the extract includes epimesembranol and any one or more of the alkaloids selected from the group consisting of sceletium A4 and its isomers, Δ7-mesembrenone, mesembrenol, mesembranol, mesembrenone, mesembrine, O-demethylmesembrenone, O-demethylmesembrine, dihydrojoubertamine, O-acetylmesembrenol and epimesembrenol.

In one embodiment, the extract includes epimesembrenol and any one or more of the alkaloids selected from the group consisting of sceletium A4 and its isomers, epimesembranol, Δ7-mesembrenone, mesembrenol, mesembranol, mesembrenone, mesembrine, O-demethylmesembrenone, O-demethylmesembrine, dihydrojoubertamine and O-acetylmesembrenol.

In one embodiment, the extract includes O-demethylmesembrine and any one or more of the alkaloids selected from the group consisting of sceletium A4 and its isomers, epimesembranol, Δ7-mesembrenone, mesembrenol, mesembranol, mesembrenone, mesembrine, O-demethylmesembrenone, dihydrojoubertamine, O-acetylmesembrenol and epimesembrenol.

In some embodiments, the combined content of mesembrenol, mesembranol, epimesembranol, O-acetyl-mesembrenol and epimesembrenol is at least about 5% (w/w), at least about 10% (w/w), at least about 20% (w/w), at least about 30% (w/w), at least about 40% (w/w), at least about 50% (w/w) or at least about 55% (w/w) of the total alkaloid content of the extract.

As used herein, the term “total alkaloid content” is intended to refer to the total content (TAC) of all the alkaloids in the extract which are detectable by LC-MS (the limit of detection (LOD) of LC-MS is 0.00001% (w/w)).

The composition can be used in medicine, such as for preventing, treating or reducing depression, anxiety, stress, stress-induced fatigue or mood, or diseases, disorders or conditions caused by these (e.g. panic attacks or seasonal mood (or affective) disorder), or combinations thereof. The composition can also be used for enhancing cognitive functioning or mood. A person suffering from (or at risk of suffering from) any of these disorders may be treated with an effective amount of the composition.

A composition of the type described herein can be used in the manufacture of a medicament, nutraceutical, natural health supplement, herbal tea, nutritive supplement or other phytopharmaceutical product. The composition, medicament, supplement or product can be used for treating, reducing or preventing anxiety, depression, stress or stress-induce fatigue, or for improving low mood or cognitive functioning.

The composition can be used in the manufacture of a medicament for treating, reducing or preventing diseases or conditions selected from the group consisting of depression, anxiety, stress, stress-induced fatigue and mood disorders, or diseases, disorders or conditions caused by these.

Treating a subject for any of the above diseases, disorders or conditions comprises administering a therapeutically effective amount of the composition to the subject (typically a human). The required dosage of the total alkaloid of the composition can vary depending on the mode of administration, the condition to be treated, the weight of the subject, and so forth. An indicated daily dosage of the total alkaloids of the composition in humans is in the range of from about 5 μg to 1 gram, such as from about 500 μg to 750 μg. The composition can be administered in a single daily dose or in divided doses, for example two or three doses a day. Suitable unit dosage forms comprise from about 5 μg to 1 gram, from about 100 μg to about 500 μg of the composition.

The composition can be formulated in the form of a pharmaceutical composition according to a method known in the art, e.g. by mixing with one or more pharmaceutically acceptable carriers or diluents.

In one embodiment, the composition includes a B-vitamin complex to facilitate the detoxification and excretion of waste products, support neurotransmitter production and enhance brain functioning. The vitamin B complex comprises thiamine (vitamin B1), riboflavin (vitamin B2), niacin (vitamin B3), pantothenic acid (vitamin B5), pyroxidine (vitamin B6), biotin (vitamin B7), folic acid (vitamin B9) and cobalamin (vitamin B12).

Vitamin D is also typically included in the composition, as vitamin D deficiency is associated with depression and low mood.

Additionally, green rooibos extract will typically also be incorporated as it has been shown to exert potent anti-oxidant activity and may therefore help combat depression by decreasing the oxidative stress and associated inflammation which are characteristics of depression. The antioxidant activity will also act as a natural preservative for the product.

The composition can be formulated for oral administration, such as in the form of a syrup, an aqueous-ethanolic tincture, a powder, a herbal tea, a tablet, a capsule, a softgel, an oral spray, a gum or a wafer.

The phytochemical profile of Sceletium plants includes a number of alkaloids. These alkaloids can be divided into the follow classes:
alkaloids of the chemical class “mesembrine”, including mesembrine, mesembranol, Δ7-mesembrenone, mesembrenone, mesembrenol, 6-epimesembranol, epimesembranol, 4′-O-demethylmesembrenone, 6-epimesembrenol, epimesembrenol, 4′-O-demethylmesembrenone isomer, O-acetylmesembrenol, 4′-O-demethylmesembrenol and Δ-7,4′-O-demethylmesembrenol;
alkaloids of the chemical class “joubertamine”, including dihydrojoubertiamine, O-methyldehydrojoubertiamine and 4-(3,4-dimethyoxyphenyl)-4-[2-acetylmethlamino)ethyl]cyclohexanone; and
alkaloids of the class “sceletium A4”, including sceletium A4 and its isomers.

Of these classes, only mesembrenone, mesembrenol and mesembrine, which are believed to be the major alkaloids and those responsible for the psychoactive and mood-enhancing properties of S. tortuosum, have hitherto been studied for their pharmacological properties. There is a lack of information about the pharmacological activity of other mesembrine-type alkaloids (for example O-demethylmesembrenone, epimesembrenol, Δ-O-demethylmesembrenol, O-acetylmesembranol etc), as well as other classes of alkaloids such as joubertiamine and the Sceletium A4 classes.

The applicant has now found that several other alkaloids that are found in some Sceletium plants may also present with therapeutic value. These alkaloids have generally been regarded as being “minor” alkaloids with little or no therapeutic activity due to their lower concentration or absence in many Sceletium plants. However, the applicant's findings surprisingly indicate that some or all of these “minor” alkaloids are present in appreciable amounts (i.e. having a content of greater than about 2% (w/w), greater than about 0.5% (w/w) or greater than or equal to about 1% (w/w) of the total alkaloid content of the extract), and may contribute to the pharmacological properties of Sceletium.

Structures and molecular properties of some of the alkaloids which have been identified in Sceletium plants are shown in Table 1.

TABLE 1
Structures and molecular properties of alkaloids found in Sceletium plants

| | |
|---|---|
| (−)- Mesembrine | |
| 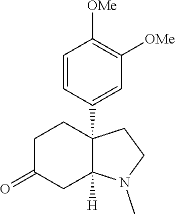 [SMILES: COc1ccc([C@@]23CCC(=O)C[C@@H]2N(C)CC3)cc1OC] | Chemical Formula: C₁₇H₂₃NO₃ Exact Mass: 289.1678 Molecular Weight: 289.3694 m/z: 289.1678 (100.0%), 290.1711 (18.4%), 291.1745 (1.6%) Elemental Analysis: C, 70.56; H, 8.01; N, 4.84; O, 16.59 |
| Mesembrenone | |
| 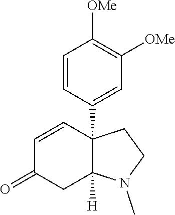 [SMILES: COc1ccc([C@@]23C=CC(=O)C[C@@H]2N(C)CC3)cc1OC] | Chemical Formula: C₁₇H₂₁NO₃ Exact Mass: 287.1521 Molecular Weight: 287.3535 m/z: 287.1521 (100.0%), 288.1555 (18.4%), 289.1589 (1.6%) Elemental Analysis: C, 71.06; H, 7.37; N, 4.87; O, 16.70 |
| D7-Mesembrenone | |
| 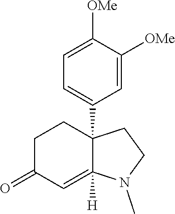 | Chemical Formula: C₁₇H₂₁NO₃ Exact Mass: 287.1521 Molecular Weight: 287.3535 m/z: 287.1521 (100.0%), 288.1555 (18.4%), 289.1589 (1.6%) Elemental Analysis: C, 71.06; H, 7.37; N, 4.87; O, 16.70 |
| Mesembrenol | |
| 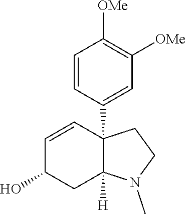 [SMILES: COc1ccc([C@@]23C=C[C@@H](O)C[C@@H]2N(C)CC3)cc1OC] | Chemical Formula: C₁₇H₂₃NO₃ Exact Mass: 289.1678 Molecular Weight: 289.3694 m/z: 289.1678 (100.0%), 290.1711 (18.4%), 291.1745 (1.6%) Elemental Analysis: C, 70.56; H, 8.01; N, 4.84; O, 16.59 |
| epimesembranol | |
| 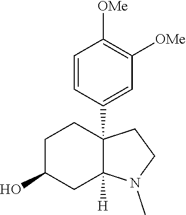 [SMILES: COc1ccc([C@@]23CC[C@H](O)C[C@@H]2N(C)CC3)cc1OC] | Chemical Formula: C₁₇H₂₅NO₃ Exact Mass: 291.1834 Molecular Weight: 291.3853 m/z: 291.1834 (100.0%), 292.1868 (18.4%), 293.1902 (1.6%) Elemental Analysis: C, 70.07; H, 8.65; N, 4.81; O, 16.47 |
| Epimesembrenol | |
| 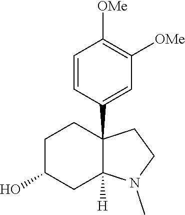 [SMILES: COc1ccc([C@]23CC[C@@H](O)C[C@@H]2N(C)CC3)cc1OC] | Chemical Formula: C₁₇H₂₃NO₃ Exact Mass: 289.17 Molecular Weight: 289.37 m/z: 289.17 (100.0%), 290.17 (18.8%), 291.17 (2.3%) Elemental Analysis: C, 70.56; H, 8.01; N, 4.84; O, 16.59 |
| dihydrojoubertiamine | |
| 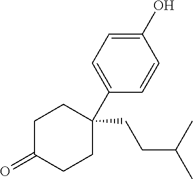 | Chemical Formula: C₁₆H₂₃NO₂ Exact Mass: 261.1729 Molecular Weight: 261.3593 m/z: 261.1729 (100.0%), 262.1762 (17.3%), 263.1796 (1.4%) Elemental Analysis: C, 73.53; H, 8.87; N, 5.36; O, 12.24 |
| O-methyldehydrojoubertiamine | |
| 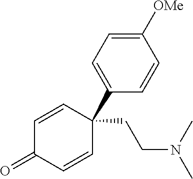 | Chemical Formula: C₁₇H₂₁NO₂ Exact Mass: 271.1572 Molecular Weight: 271.3541 m/z: 271.1572 (100.0%), 272.1606 (18.4%), 273.1639 (1.6%) Elemental Analysis: C, 75.25; H, 7.80; N, 5.16; O, 11.79 |
| 4′-O-methylmesembrenone | |
| 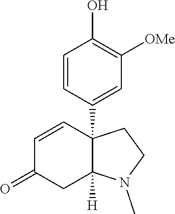 [SMILES: COc1cc([C@@]23C=CC(=O)C[C@@H]2N(C)CC3)ccc1O] | Chemical Formula: C₁₆H₁₉NO₃ Exact Mass: 273.1365 Molecular Weight: 273.3270 m/z: 273.1365 (100.0%), 274.1398 (17.3%), 275.1432 (1.4%) Elemental Analysis: C, 70.31; H, 7.01; N, 5.12; O, 17.56 |
| 4′-O-demethylmesembranol | |
| 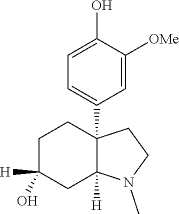 [SMILES: COc1cc([C@@]23CC[C@@H](O)C[C@@H]2N(C)CC3)ccc1O] | Chemical Formula: C₁₆H₂₃NO₃ Exact Mass: 277.1678 Molecular Weight: 277.3587 m/z: 277.1678 (100.0%), 278.1711 (17.3%), 279.1745 (1.4%) Elemental Analysis: C, 69.29; H, 8.36; N, 5.05; O, 17.31 |
| 4′-O-demethylmesembrenol | |
| 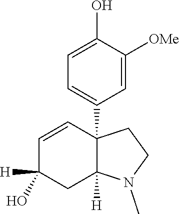 [SMILES: COc1cc([C@@]23C=C[C@@H](O)C[C@@H]2N(C)CC3)ccc1O] | Chemical Formula: C₁₆H₂₁NO₃ Exact Mass: 275.1521 Molecular Weight: 275.3428 m/z: 275.1521 (100.0%), 276.1555 (17.3%), 277.1589 (1.4%) Elemental Analysis: C, 69.79; H, 7.69; N, 5.09; O, 17.43 |
| O-Acetylmesembrenol | |
| 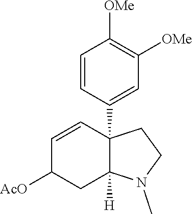 [SMILES: COc1ccc([C@@]23C=CC(OC(C)=O)C[C@@H]2N(C)CC3)cc1OC] | Chemical Formula: C₁₉H₂₅NO₄ Exact Mass: 331.1784 Molecular Weight: 331.4061 m/z: 331.1784 (100.0%), 332.1817 (20.5%), 333.1851 (2.0%) Elemental Analysis: C, 68.86; H, 7.60; N, 4.23; O, 19.31 |
| Sceletium alkaloid A4 | |
| 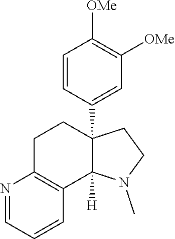 [SMILES: COc1ccc([C@@]23CCc4ncccc4[C@@H]2N(C)CC3)cc1OC] | Chemical Formula: C₂₀H₂₄N₂O₂ Exact Mass: 324.1838 Molecular Weight: 324.4168 m/z: 324.1838 (100.0%), 325.1871 (21.6%), 326.1905 (2.2%) Elemental Analysis: C, 74.04; H, 7.46; N. 8.64; O, 9.86 |

In order to prepare the composition of the invention, plant material that has the alkaloid profile as claimed herein must be selected. This can be achieved by analysing the alkaloid profiles of individual Sceletium plants after extracting the alkaloids using an extraction method and performing LC-MS analysis, such as by the methods described in the examples below. Alternatively, a person of skill in the art will understand that other extraction methods could also be used.

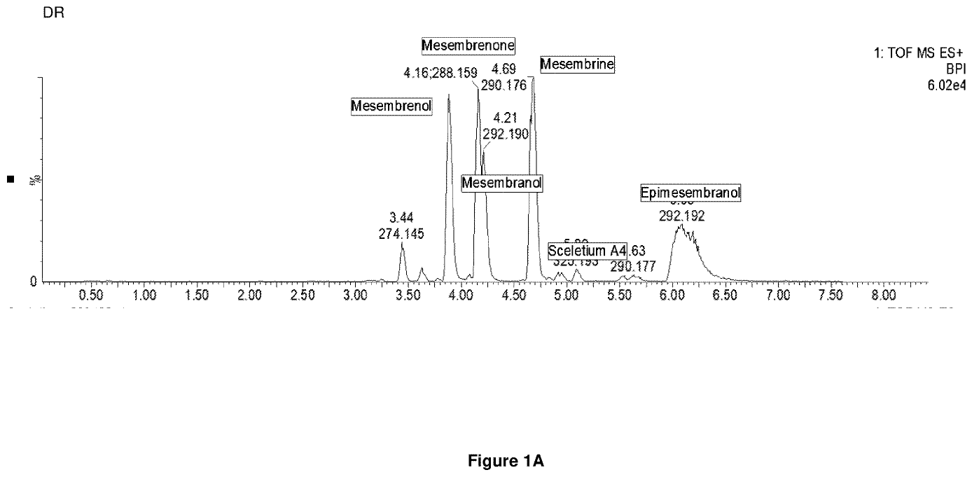

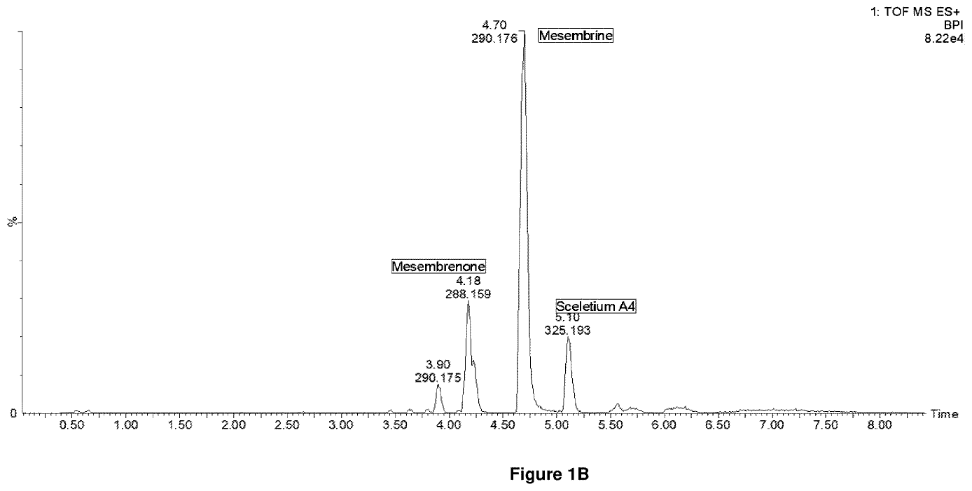

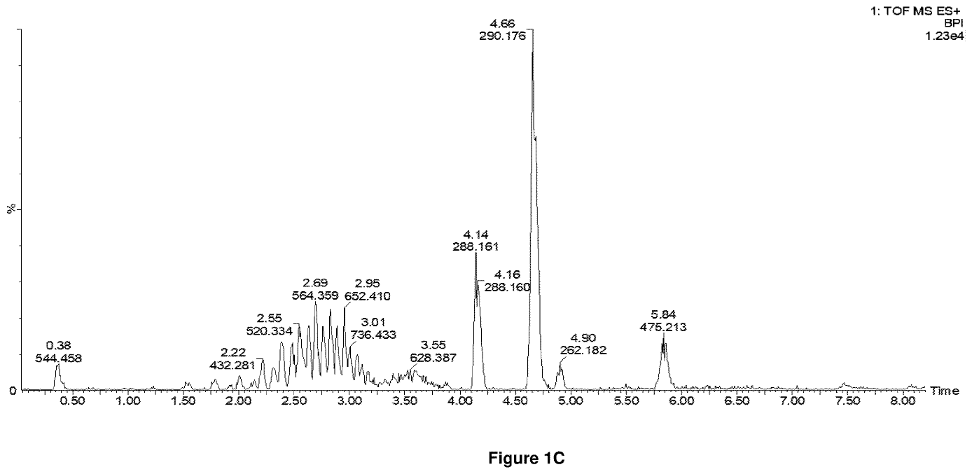

FIG. 1 shows a typical LC-MS chromatogram of an extract with peaks for sceletium A4 1, sceletium A4 2 and epimesembranol. The LC-MS chromatogram has been annotated to show the MS spectra and chemical structures of sceletium A4 1, sceletium A4 2 and epimesembranol.

Plants identified and selected for having the desired total alkaloid profile may be kept as propagation material and as seed stock. Selected plants can be grown, such as under Sceletium growing conditions that are described in the art. The aerial parts of the plant can be harvested without any flowers or seed capsules and washed in water. Fresh or frozen plant material can then be macerated in a blender to produce a “plantsap”, which can then be centrifuged for 30 min. The supernatant can be transferred to a fresh centrifuge tube and freeze-dried. The alkaloids are taken up in ethanol. This ethanolic solution can be stored at 4° C., and is typically used for one week and then discarded.

While the method described herein can be used to produce the extract for the composition of the present invention, any process which can be performed on an industrial scale (for example vacuum drying instead of freeze-drying) could also be used for obtaining the composition of the present invention. Such processes are well-known to those of skill in the art.

Additives such as magnesium, vitamin B-complex, vitamin D3 and green rooibos extract can be added to the composition to provide an intended daily dose as shown in Table 2.

TABLE 2
Doses of additional supplements to be combined with Sceletium extract.

| | | |
|---|---|---|
| Vitamin B complex | Thiamine (vitamin B1) | 1.1 mg (RDA)-100 mg |
| | Riboflavin (vitamin B2) | 1.1 mg (RDA)-100 mg |
| | Niacin (vitamin B3) | 14 mg-35 mg |
| | Pantothenic acid (vitamin B5) | 5 mg-200 mg |
| | Pyroxidine (vitamin B6) | 1.3 mg-100 mg |
| | Biotin (vitamin B7) | 30 ug (RDA)-500 ug |
| | Folic acid (vitamin B9) | 400 ug (DFE)-500 ug |
| | Cobalamin (vitamin B12) | 2.4 ug (RDA)-100 ug |
| Vitamin D₃ | Cholecalciferol | 600-1000 IU |
| Magnesium Glycinate | | RDA - 310 mg-420 mg |
| Green Rooibos Extract | Aspalathus linearis | 25-90 mg/kg per day (in vivo |
| | | dose range) |

The invention will now be described in more detail with reference to the following non-limiting examples.

### EXAMPLES

Samples from different species of Sceletium (including S. tortuosum, S. strictum, S. emarcidum, and S. rigidum) were harvested from the Succulent Karoo in South Africa. Samples from different ecotypes of S. tortuosum were also obtained. The plants were analyzed through liquid chromatography-mass spectrometry (LC-MS), using MSE fragmentation as a tool for chemical identification.

As used herein, the term “ecotype” (sometimes called ecospecies) describes a genetically distinct geographic variety or population within a species, which is genotypically adapted to specific environmental conditions. Ecotypes exhibit phenotypic differences (such as in morphology or physiology) stemming from environmental heterogeneity.

### Phytochemical Extraction

The plant material was dried over silica in a sealed plastic bag, in darkness at room temperature. Leaves were ground to a fine powder using liquid nitrogen using a mortar and pestle. For each extraction, the sample powder was weighed and transferred into a 2 mL Eppendorf® Safe-Lock microcentrifuge polypropylene tube. Samples were extracted using methanol as an extraction solvent, maintaining a concentration of 50 mg/mL of plant sample to solvent. Samples were vortexed (20 s), sonicated (20 min) (Branson 50/60 Hz, Branson Cleaning Equipment Company, USA), and benchtop centrifuged (10 min) (Hermle Z160m, 3000×g). The supernatant was aliquoted (1 mL) into autosampler vials for metabolite analysis. All samples were stored at 5° C. and analysed within 24 h of being extracted.

### Ultra-High Performance Liquid Chromatography-Mass Spectrometry Instrumentation

Chemical analysis was executed on an Acquity ultra-high-performance liquid chromatography (UHPLC) system (Waters Corporation, USA) coupled to a Waters Acquity photodiode array (PDA) UV detector (230-500 nm) and Synapt G2 HDMS qToF mass spectrometer (Waters Corporation, USA). Separation of metabolites was performed on a UPLC™ BEH C₁₈ column (2.1×100 mm, i.d., 1.7 μm particle size, Waters). Electrospray ionisation was applied in the positive mode (ESI+) using a Z-spray source with the following ionisation conditions; 15V cone voltage, 2.5 kV capillary voltage, 120° C. source temperature, 50 L/hr cone gas flow. Nitrogen at 650 L/hr was used as the desolvation gas and a desolvation temperature of 275° C. was applied. A Waters Acquity UPLC Binary Solvent Manager delivered the mobile phase solvents at a flow rate of 0.3 mL/min. The gradient was initiated at 100% 0.1% ammonium hydroxide (Solvent A) and held for 5 min, followed by a linear gradient transition to 100% acetonitrile containing 0.1% ammonium hydroxide (Solvent B) over 2 min, where was held for 3 min, followed by a return to 100% A over 0.1 min, remaining here for 2.9 min to re-equilibrate the column and giving a total run time of 13 min. Ammonium hydroxide was used as a mobile phase as it was found to induce the best separation of Sceletium-derived alkaloids when the method was being developed. At high pH, the alkaloids possess a neutral charge, and this gave better chromatographic peak shapes and retention times. However, a low pH results in the alkaloids being positively charged and are thus not well retained on the reverse phase system used in this study. Mass spectral data were attained using an 160-1500 Da range window. Data were centroided during acquisition and the LockSpray™ module was used to ensure mass accuracy with leucine encephalin as reference. A 3 μL sample injection volume was used. Methanol (HPLC grade; UV cut-off 215 nm) and acetonitrile (UV cut-off 200 nm) (ROMIL Ltd., Microsep, South Africa) were used for sample preparation and for the mobile phase. Dilution of reagents in all cases used ultrapure analytical grade Type 1 water (Milli-Q®, Merck, Darmstadt, Germany).

### Data Interpretation and Analysis

### Quantitative Chemical Analysis

Two independent LC-MS analyses were conducted, and the datasets were combined irrespective of the LC-MS run. From the combined dataset, feature picking was conducted manually with eighteen different features that occurred between retention time 3.00 min and retention time 7.00 min. These eighteen features were selected based on literature sources where chemicals where identified in Sceletium species (order of elution and MS/MS fragmentation patterns), Variable Importance in Projection (VIP) scores that indicate those metabolites contributing the greatest differences amongst populations, bi-plots and loadings plots across metabolomic experiments, chemical clusters identified from spectra deconvolution in molecular networking, feature picking in MS-DIAL, as well as formula identification from elemental composition analysis based on MS1 spectra in MassLynx. Data acquisition and processing were carried out using TargetLynx™ Application Manager for MassLynx™ v4.1 software (Waters Corporation, USA) for quantitation (mg/kg DW) of compounds using the integrated peak areas of extracted mass chromatograms and mesembrine as the in-house reference standard. A m/z range of 262 to 334 and an average threshold of 1.5 min was used for the quantification of phytochemicals. Each peak was inspected, and peak areas selected manually where errors were observed in TargetLynx. Relative quantification was carried out using a concentration range of the mesembrine standard from 3.125 ppm to 100 ppm where each metabolite quantified according to this straight-line curve where volume and mass used in the extraction was also taken into account. Phytochemicals were quantified and reported as dry weight (DW) yield (mg/kg). The numerous putatively identified alkaloid metabolites of interest in samples, for which no commercial standards exist, were quantified relative to the mesembrine standard, and expressed as mesembrine equivalents, facilitating their semi-quantitative analysis and comparison. Alkaloid identification was conducted by referencing the experimental m/z, elution time, UV spectra, and elemental composition analysis (±5 ppm tolerance) to published structures, in-house reference standards data sets (mesembrenone and mesembrenol), and where possible confirmed by MSE fragmentation-PDA datasets.

### Multivariate Statistical Analysis: PCA and PLS-DA

The multivariate data analysis tool of a principal component analysis (PCA) and partial least squares discriminant analysis (PLS-DA) of the UHPLC-MS data set was used to visualise qualitative differences in chemical composition of different populations and species of Sceletium. Detection of relevant biomarker peaks and the generation of the data matrix from the large, raw UHPLC-MS chromatographic datasets was performed in MS-DIAL (v4.70). In MS-DIAL, the parameters of data collection were set to a retention time window of 1.5 min to 8.5 min, an MS1 mass range of 120 Da to 1200 Da and an MS/MS mass range of 40 Da to 1000 Da. Peak detection was set to an amplitude of 1000 and a retention time tolerance of 0.05 min. The resulting pre-processed dataset matrix was exported to an excel workbook format (.xlsx) and then converted to a comma delimited file format (.csv) that was then analysed using MetaboAnalyst (http://metaboanalvst.ca). Peak intensity data were filtered using median intensity to remove variables that were unlikely to be of use when modelling the data. Sample normalization was done by sum, to adjust for systematic differences among samples. Data were log transformed and auto scaled (mean-centered and divided by the standard deviation of each variable). Score plots and loading plots were further used to identify key metabolites contributing to the differences between and within populations. These metabolites were then tentatively identified based on the MSE fragmentation patterns, literature sources, molecular masses and predicted molecular formulae. The selection of metabolites was then used in further metabolomic experiments. Heatmaps with hierarchal cluster analyses (HCA) were also created from the phytochemicals found in plant populations, and the study species. Model validation with R2, Q2 and accuracy scores were conducted using MetaboAnalyst. Classification and cross-validation were performed in MetaboAnalyst using the wrapper function in the caret package which was run using R-code on the MetaboAnalyst website.

### Univariate Statistical Analyses

A one-way analysis of variance (ANOVA) for all quantitative data was performed using GraphPad Prism version 8.0.1 for Windows (GraphPad Software, San Diego, California USA (www.qraphpad.com). Prior to this, D'Agnostino-Pearson omnibus normality test was conducted to test if the data conformed to a normal (Gaussian) distribution. Data were also manually assessed for outliers and where present these were removed on a case-by-case basis. To separate the means, multiple comparisons were conducted as a post-hoc test, for pairwise comparisons. A post-hoc Tukey test was thus used to test the statistical hypothesis for normally distributed data. In cases where data did not conform to assumptions of normality, a non-parametric test using Kruskal-Wallis analysis was regarded as being most appropriate. Descriptive statistics using boxplots or stacked column charts were also employed to visualise the data.

### Feature-Based Molecular Networking

Raw data files were converted to .mzML files using the MS convert tool (ProteoWizard version 3.0.1904) and loaded onto MS-DIAL where data from SWATH-MS² (data independent LC-MS² acquisition) was employed. Retention time tolerance and MS1 tolerance were set to 0.1 min and 0.02 Da, respectively. Data from the UHPLC-MS^{E} analysis of Sceletium populations was processed in MS-Dial to perform feature picking. This process enabled the identification of minor chemical constituents as well as co-eluting compounds. Once peak detection and MSE deconvolution was complete the MSE spectra were exported as an .mgf file and qualitative data as a .csv file which were both used to construct molecular networks on the Global Natural Products Social Molecular Networking (GNPS) platform (https://qnps.ucsd.edu). Parameters for feature-based molecular networking were set as follows: precursor ion mass tolerance, 0.05; min pair cosine, 0.7; network topK, 10; maximum connected component size, 100; minimum matched product ions, 3; minimum cluster size, 6. The feature-based molecular network was constructed and visualized in CytoScape (version 3.7.0). Once data was opened in CytoScape, maps were altered with regards to styles of nodes, Edge and Network. Nodes were annotated and scaled with respect to parent ion masses. Distance between nodes was set to represent MS2 spectrum similarity. Networks were overlaid with geographic location and visualised with Passthrough mapping settings. Label colours, position and size were altered for best visual appearance. For retention time networks, nodes were coloured by retention time using Passthrough mapping.

### In Vivo Testing

Ninety randomly selected C57bl/6 mice (age 10-18 weeks) were bred and divided into 5 experimental groups (n=12 per group equally distributed between sexes). Mice were housed in same-sex cages (six animals per cage) for the duration of the study. Each mouse was exposed to 35 days of uninterrupted oral administration of either a normal water or 5% EtOH control, or one of the three Sceletium extracts being 3K, DR or industry sample. From exposure day 31, animals underwent sequential behavioural tests over four days after which they were euthanised on exposure day 35. Subsequently, the whole brain was removed, and the prefrontal-cortices (PFC), hippocampi (HIP) and striatal tissue (STR) dissected. The levels of the neurotransmitters serotonin, noradrenaline, and dopamine, and gamma-aminobutyric acid (GABA) were measured.

### Drug Administration

Since crude plant extracts vary with respect to alkaloid content, it was necessary to standardise the administered dose based on total alkaloid content (TAC). Thus the same TAC was administered for each of the three extracts, although the formulas differed based on the types of alkaloids they contained.

The total alkaloid content (TCA) of the extracts was quantified as follows:
Industry sample: 0.7% TAC
3K sample: 0.94% TAC
DR sample: 0.46% TAC

The extracts were diluted into ethanol and the volume was adjusted to give a dose of 10 mg/kg (1.4 μg TAC per mouse) in 5% EtOH.

### Results and Discussion

### Chemical Profiling of Sceletium Populations

Methanolic extracts were analyzed in a LC-QToF-MS system prior to use of a semi-supervised metabolomic analysis. Metabolomic analyses were conducted to compare all populations of Sceletium.

Seventeen alkaloids of the mesembrine-type were identified and analysed in the extracts (Table 3).

TABLE 3
Alkaloids from Sceletium species collected from the wild

| Network | | Alkaloid | m/z | Retention | Molecular |
|---|---|---|---|---|---|
| number | Compound name | chemical class | (M + H¹) | time (min) | formula* |
| 1 | mesembrine | Mesembrine | 290.1757 | 5.10 | C17H24NO3 |
| 3 | mesembranol | Mesembrine | 292.1913 | 4.61 | C17H26NO3 |
| | Δ7-mesembrenone | Mesembrine | 288.1600 | 4.53 | C17H22NO3 |
| | mesembrenone | Mesembrine | 288.1596 | 4.82 | C17H22NO3 |
| 4 | mesembrenol | Mesembrine | 290.1763 | 4.54 | C17H24NO₃ |
| | O-methyldehydrojoubertiamine | Joubertiamine | 272.1674 | 4.54 | C17H22NO2 |
| 7 | sceletium A4 | Sceletium A4 | 325.1914 | 5.47 | C20H25N2O2 |
| | sceletium A4 isomer | Sceletium A4 | 325.1910 | 5.69 | C20H25N2O2 |
| 8 | epimesembranol | Mesembrine | 292.1884 | 6.57 | C17H26NO3 |
| 10 | 4′-O-demethylmesembrenone | Mesembrine | 274.1442 | 3.89 | C16H19NO3 |
| 11 | epimesembrenol | Mesembrine | 290.1748 | 5.35 | C17H24NO3 |
| 12 | dihydrojoubertiamine^{a} | Joubertiamine | 262.1805 | 5.47 | C16H24NO2 |
| 13 | 4′-O-demethylmesembrenone | Mesembrine | 274.1446 | 4.11 | C16H19NO3 |
| | isomer | | | | |
| 14 | 4-(3,4-dimethyoxyphenyl)-4-[2- | Joubertiamine | 334.2020 | 6.60 | C19H28NO4 |
| | acetylmethlamino)ethyl]cyclohexanone | | | | |
| 15 | O-acetylmesembrenol | Mesembrine | 332.1875 | 5.98 | C19H26NO4 |
| 16 | 4′-O-demethylmesembrenol | Mesembrine | 276.1597 | 4.17 | C16H22NO3 |
| 18 | Δ-7,4′-O-demethylmesembrenol | Mesembrine | 276.1602 | 4.09 | C16H22NO3 |

*refers to [M + H]⁺ ion detected by LC-MS.

The average concentration [expressed as mg/kg (w/w) and % total alkaloids (% TA)] for each of the 17 alkaloids with standard deviations is shown in Table 4.

TABLE 4
Averages of Sceletium alkaloids found between
all the species and ecotypes tested.

| | | mg/kg (w/w) | | % TAC | % |
|---|---|---|---|---|---|
| | | Ave | SD | AVE | SD |
| 1 | mesembrine | 333.7 | 157.6 | 32.5 | 16.8 |
| 2 | mesembranol | 140.9 | 76.76 | 14.4 | 15.7 |
| 3 | Δ7 mesembrenone | 131.1 | 54.59 | 15.1 | 5.67 |
| 4 | mesembrenone | 134.5 | 132.2 | 7.81 | 8.41 |
| 5 | mesembrenol | 199.9 | 184.6 | 13.7 | 17.2 |
| 6 | O-methyljoubertiamine | 4.75 | 4.29 | 0.20 | 0.18 |
| 7 | sceletium A4 | 20.84 | 14.62 | 1.2 | 0.93 |
| 8 | sceletium A4 isomer | 171.6 | 59.03 | 6.08 | 2.59 |
| 9 | epimesembranol | 120.6 | 15.30 | 7.67 | 8.11 |
| 10 | 4′-O-demethylmesembrenone | 1.2 | 0.94 | 0.26 | 0.45 |
| 11 | epimesembrenol | 33.89 | 15.30 | 8.40 | 2.43 |
| 12 | dihydrojoubertiamine | 12.14 | 9.54 | 0.89 | 0.82 |
| 13 | 4′-O-demethylmesembrenone isomer | 35.25 | 32.78 | 7.70 | 12.12 |
| 14 | 4-(3-methoxy-4-hydroxyphenyl)-4-(2-acetyl- | 4.77 | 6.12 | 0.54 | 0.78 |
| | methylaminoethyl)cyclohexadienone | | | | |
| 15 | O-acetylmesembrenol | 0.92 | 0.46 | 0.10 | 0.04 |
| 16 | 4′-O-demethylmesembrenol | 19.40 | 27.07 | 8.51 | 14.77 |
| 17 | Δ7 4′-O-demethylmesembrenol | 56.24 | 27.68 | 13.98 | 6.80 |

The metabolomic profiles were found to vary significantly between species and also between ecotypes of the same species. Two S. tortuosum ecotypes (designated herein as “3K” and “DR”) were identified as being of particular interest because of their alkaloid composition, and these were compared to a commercially available S. tortuosum extract (referred to herein as the industry sample) (Table 5). The extract from DR contained a high concentration of mesembrenol (1171 mg/kg, 24% TA) compared to the 3K extract (140 mg/kg, 1.6% TA) and the industry sample (which did not contain mesembrenol) (Table 4). The DR sample also contained other lesser-known alkaloids not present in the industry sample, namely 0-demethylmesembrine, epimesembranol, epimesembrenol and mesembrenol].

TABLE 5
Alkaloid concentrations in Sceletium extracts from DR, 3K, and the industry sample.

| | | Industry | | 3K | | DR | |
|---|---|---|---|---|---|---|---|
| | | % TAC | ug/mL | % TAC | mg/kg | % TAC | mg/kg |
| 1 | Δ7-mesembrenone | 1.41 | 12.2 | 0.280 | 25.1 | 0.170 | 8.23 |
| 2 | mesembrenol | — | — | 1.58 | 140 | 24.0 | 1171 |
| 3 | mesembranol | 1.22 | 10.6 | 3.72 | 330 | 9.80 | 478 |
| 4 | mesembrenone | 24.6 | 213 | 13.1 | 1162 | 25.4 | 1239 |
| 5 | mesembrine | 66.9 | 579 | 69.7 | 6183 | 10.2 | 496 |
| 6 | sceletium A4 1 | 0.01 | 0.09 | 0.930 | 82.7 | 5.40 | 263 |
| 7 | epimesembranol | — | — | 2.07 | 184 | 21.3 | 1037 |
| 8 | sceletium A4 2 | 0.250 | 2.20 | 7.58 | 672 | 1.33 | 65.0 |
| 9 | O-demethylmesembrenone | 0.430 | 3.71 | 0.230 | 20.5 | 0.900 | 43.7 |
| 10 | O-demethylmesembrine | — | — | 0.230 | 20.6 | 0.310 | 15.3 |
| 11 | Dihydrojoubertamine | 5.06 | 43.8 | 0.210 | 18.9 | 0.520 | 25.1 |
| 12 | O-acetylmesembrenol | 0.060 | 0.53 | 0.090 | 8.4 | 0.120 | 5.86 |
| 13 | epimesembrenol | — | — | 0.250 | 22.3 | 0.530 | 25.7 |
| 15 | 4-O-demethylmesembrenol | — | — | — | — | — | — |

When looking at clusters of alkaloids in the three S. tortuosum extracts of interest (Table 6), the total alkaloid content also varied. For example, the Industry and 3K samples were high in mesembrine and low in mesembrenone, whereas the DR sample was low in mesembrine and higher in mesembrenone. This is reflected in the mesembrenone: mesembrine ratios which are 0.4 vs 0.2 vs 2.50 for the industry vs 3K vs DR. The DR sample on the other hand is particularly high in the mesembrenol cluster of alkaloids (mesembrenol, mesembranol and epimesembranol) of 55.8 vs 7.7 vs 1.3 for DR versus 3K versus industry. Both 3K and DR samples had considerable quantities of the minor alkaloids Sceletium A4 1 and 2 compared to the industry sample (8.5 vs 6.7 vs 0.3). This data highlights the large variations in alkaloid profiles between the industry sample and the 3K and DR Sceletium samples.

TABLE 6
Clusters of alkaloid classes in Sceletium extracts
from DR, 3K, and the Industry sample.

| % TAC | | | |
|---|---|---|---|
| Industry | 3K | DR | Classes of alkaloids |
| 66.9 | 69.7 | 10.2 | mesembrine |
| 24.6 | 13.1 | 25.4 | mesembrenone |
| 26.5 | 13.6 | 26.5 | mesembrenone + Δ7-mesembranone + |
| | | | O-demethylmesembrenone |
| 24.6 | 14.7 | 49.5 | mesembrenol + mesembrenone |
| 1.3 | 7.7 | 55.8 | mesembrenol + mesembranol + |
| | | | epimesembranol + O-acetyl- |
| | | | mesembrenol + epimesembrenol |
| 0.4 | 0.2 | 2.5 | ratio: mesembrenone/mesembrine |
| 91.6 | 82.8 | 35.6 | mesembrenone + mesembrine |
| 66.9 | 69.9 | 10.5 | mesembrine + O-demethylmesembrine |
| 0.3 | 8.5 | 6.7 | sceletium A4 1 + 2 |

Thus the concentrations and ratios of the mesembrine-type alkaloids in different Sceletium ecotypes varies. In addition, some of these alkaloids, which may be considered as “minor” are present in amounts which are greater than 1% (i.e. Sceletium A4, Table 4).

The effects of the DR and 3K S. tortuosum extracts on anxiety and depression in a mouse model were investigated and compared to the industry sample. The study looked at the effects of S. tortuosum extracts containing different alkaloid content on anxiety- and depressive-like behaviours in C57bl/6 mice and measured the levels of the neurotransmitters serotonin, noradrenaline, dopamine and gamma-aminobutyric acid (GABA).

### Serotonin Levels

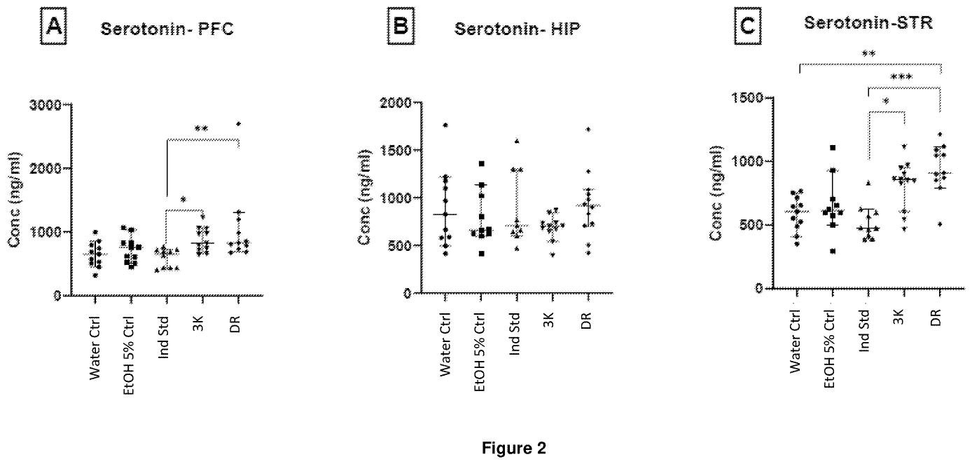

Serotonin is a neurotransmitter that regulates mood, and low serotonin levels may result in a low mood and depression. Depression is therefore often treated with selective serotonin reuptake inhibitors (SSRIs) to increase serotonin levels and improve mood. The results of this study indicated that treatment with the 3K and DR extracts increased serotonin levels in the prefrontal cortex and the striatum compared to the industry sample, which showed no effect compared to the controls. In the striatum, the DR sample increased the serotonin levels significantly compared to the water control (p>0.01), whereas the industry sample had no effect (FIG. 2). The significant differences between the DR or 3K extracts and the industry sample may be due to the industry sample behaving more like a control, but with narrower standard deviations within the group. Thus, the DR and 3K S. tortuosum extracts may have an effect at reducing depression, as they both significantly increased serotonin levels, with the DR sample showing the greater effect.

### Noradrenaline Levels

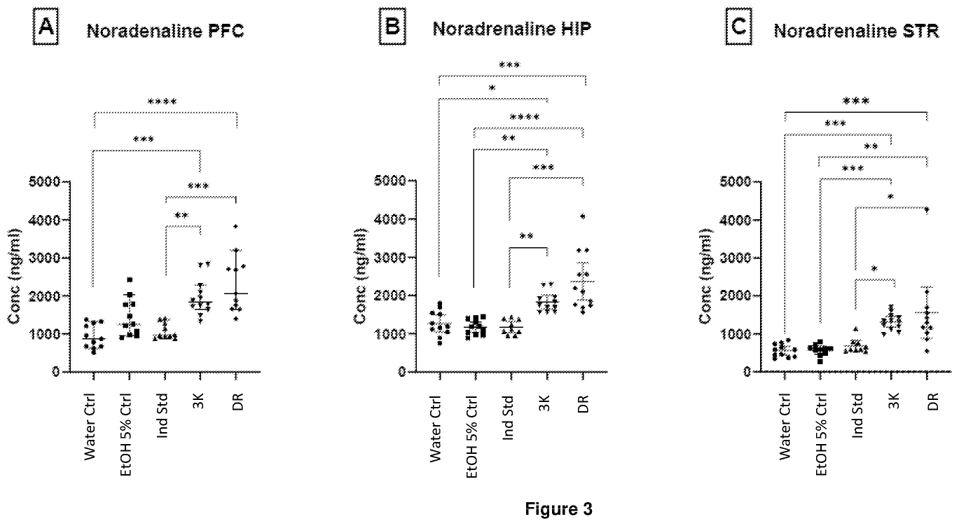

The levels of noradrenaline in the prefrontal cortex, hippocampus, and striatum were also measured at the end of the chronic study. Noradrenaline is a neurotransmitter and a hormone that plays a role in the fight-or-flight response. Health conditions resulting from low noradrenaline include anxiety, depression, attention deficit hyperactivity disorder (ADHD), sleep impairments, hypotension, and reduced concentration. Serotonin-noradrenaline reuptake inhibitors (SNRIs) are often prescribed to treat depression. The SNRIs inhibit the reuptake of serotonin and noradrenaline to increase the levels of these neurotransmitters. The results of this study indicated that treatment with the 3K and DR extracts increased noradrenaline levels 2-3 fold in all of the regions of the prefrontal cortex, hippocampus, and striatum compared to the water control in mice. All of the results were significant with DR showing the most significance [p<0.001 (PFC), p<0.005 (HIP) and p<0.005 (STR)] and with the highest increases in the prefrontal cortex and hippocampus. The industry sample had no effect on this neurotransmitter in any of the regions studied, although again, it was found to lower the variation between the mice (FIG. 3). Compared to the ethanol control, both the 3K and DR extracts significantly increased the levels of noradrenaline in the hippocampus (p<0.01 and 0.001, respectively) and striatum (p<0.005, p<0.05, respectively). Note that the vehicle ethanol control did not significantly increase the levels of noradrenaline. These strongly significant results indicate that the 3K and DR extracts may be beneficial for patients suffering from depression who display low norepinephrine levels. Both extracts showed higher activity than the industry sample.

### Dopamine Levels

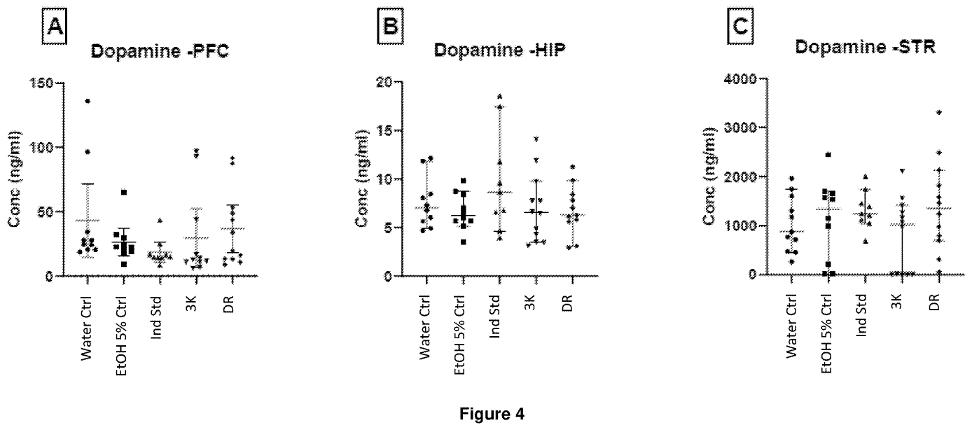

Dopamine promotes pleasure, motivation, and satisfaction, and its levels are often low in patients suffering from depression. None of the Sceletium extracts affected dopamine levels (FIG. 4).

### Gamma-Aminobutyric Acid (GABA) Levels

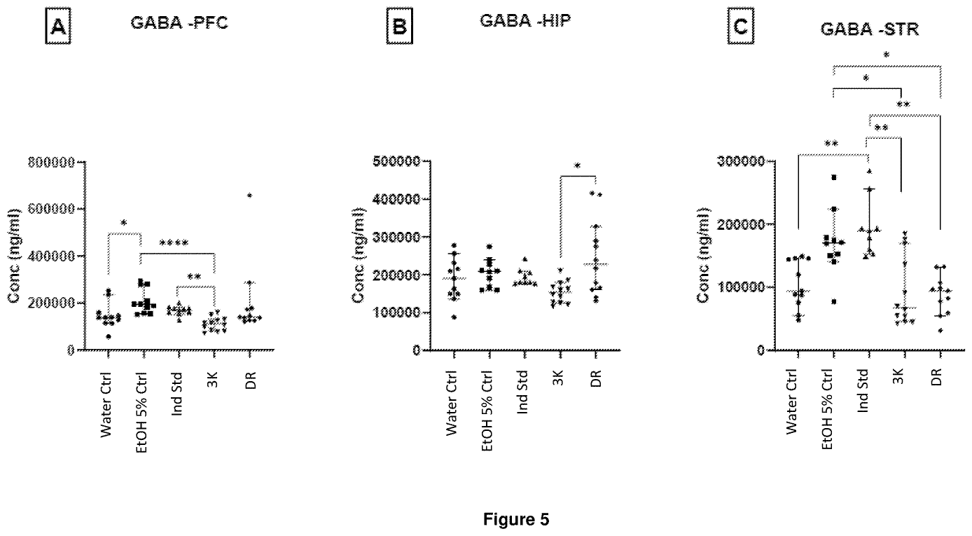

GABA is an inhibitory neurotransmitter that lessens a nerve cell's ability to receive, create, or send chemical signals to adjacent nerve cells. GABA produces a calming effect and reduces anxiety, stress, and fear. It was found that the ethanol control significantly increased GABA in the prefrontal cortex. It also appeared to increase GABA in the STR (not significant). The 3K extract (PFC and STR) and DR extract (STR) appeared to return the elevated GABA from the 5% EtOH back to basal levels (PFC). There was no effect observed in the hippocampus (FIG. 5). Thus, the DR and 3K extracts appear to counter the effect of EtOH on GABA.

Taken together, the results indicate that the alkaloid profile in Sceletium extracts varies between ecotypes within S. tortuosum. The extracts from 3K and DR specifically stand out as having unique alkaloid profiles, where 3K is high in mesembrine and low in mesembrenone, while the DR sample is high in mesembrenone and low in mesembrine. The DR sample is also particularly high in the mesembrenols (mesembrenol, mesembranol, epimesembranol, O-acetylmesembrenol and epimesembrenol), with 56% TAC compared to the 3K extract (7.7%) and industry sample (1.3%). Both the 3K and DR extracts are high in the minor alkaloids Sceletium A4 and its isomer (8.5 and 6.7%, respectively) compared to the industry sample (0.3%). Since the DR sample showed higher activity over 3K in the in vivo study, it may be that the effects are due to the presence of the high mesembrenols and/or the high mesembrenone over mesembrine. The most active sample (DR) was not high in mesembrine, which is the alkaloid commonly associated with the mood-enhancing effects of S. tortuosum.

The DR and 3K samples might exert their antidepressant functions by increased serotonin and noradrenaline levels. In contrast, the industry sample, which is a commercial product, did not significantly affect these neurotransmitters. The extracts also appeared to reverse the effects of EtOH on slightly elevated GABA. No differences in dopamine levels were observed between groups treated with any of the Sceletium extracts. These data suggest that the Sceletium samples of this study, and particularly the DR sample, may be more beneficial for individuals with low mood and depression than the industry sample that was tested.

To the best of the applicant's knowledge, this is the first published evidence of the possible involvement of alkaloids other than the mesembrine-type phytochemicals responsible for the neurological activity of this plant.

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Finally, throughout the specification and accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

1. A composition comprising an extract of a plant from the genus Sceletium,
wherein the extract comprises sceletium A4, an isomer of sceletium A4 and epimesembranol;
and wherein:
the content of each of the sceletium A4 alkaloids is not less than about 0.9% (w/w) of the total alkaloid content of the extract; and
the content of epimesembranol is not less than about 1% (w/w) of the total alkaloid content of the extract.

2. The composition of claim 1, wherein the content of sceletium A4 is not less than about 5% (w/w) of the total alkaloid content of the extract.

3. The composition of claim 1, wherein the content of the sceletium A4 isomer is not less than about 5% (w/w) of the total alkaloid content of the extract.

4. The composition of claim 1, wherein the content of epimesembranol is not less than about 7% (w/w) of the total alkaloid content of the extract.

5. The composition of claim 1, wherein the combined content of the sceletium A4 alkaloids is at least about 5%(w/w) of the total alkaloid content of the extract.

6. The composition of claim 1, wherein the extract further comprises any one or more of the alkaloids selected from the group consisting of mesembrenol, O-demethylmesembrine and epimesembrenol.

7. The composition of claim 1, wherein the extract further comprises mesembrenol, O-demethylmesembrine and epimesembrenol.

8. The composition of claim 1, wherein the extract further comprises any one or more of the alkaloids selected from the group consisting of Δ7-mesembrenone, mesembrenol, mesembranol, mesembrenone, mesembrine, O-demethylmesembrenone, O-demethylmesembrine, dihydrojoubertamine, O-acetylmesembrenol and epimesembrenol.

9. The composition of claim 1, wherein the extract further comprises the alkaloids Δ7-mesembrenone, mesembrenol, mesembranol, mesembrenone, mesembrine, O-demethylmesembrenone, O-demethylmesembrine, dihydrojoubertamine, O-acetylmesembrenol and epimesembrenol.

10. The composition of claim 8, wherein the combined content of mesembrenol, mesembranol, epimesembranol, O-acetyl-mesembrenol and epimesembrenol is at least about 5% (w/w) of the total alkaloid content of the extract.

11. The composition of claim 8, wherein the combined content of mesembrenol, mesembranol, epimesembranol, O-acetyl-mesembrenol and epimesembrenol is at least about 50% (w/w) of the total alkaloid content of the extract.

12. The composition of claim 1, wherein the content of epimesembranol is not less than about 20% (w/w) of the total alkaloid content of the extract.

13-15. (canceled)

16. A method of treating, reducing or preventing diseases or conditions selected from the group consisting of depression, anxiety, stress and stress-induced fatigue or for enhancing cognitive functioning or mood, the method comprising administering an effective amount of a composition of claim 1 to a subject in need thereof.
